(19) United States
(12) Patent Application Publication
(10) Pub. No.: US 2007/0032703 A1
(43) Pub. Date: Feb. 8, 2007

(54) Radially expansive surgical instruments for tissue retraction and methods for using the same

(76) Inventors: Meera L. Sankaran, Cupertino, CA (US); Christine Seto, Los Angeles, CA (US)

Correspondence Address: KILPATRICK STOCKTON LLP - 55461, 1001 WEST FOURTH STREET, WINSTON-SALEM, NC, 27101, US

(21) Appl. No.: 11/476,337

(22) Filed: Jun. 27, 2006

Related U.S. Application Data
(60) Provisional application No. 60/698,128, filed on Jul. 11, 2005.

(51) Int. Cl. A61B 1/32

(52) U.S. Cl. 600/208

(57) ABSTRACT

The present invention is directed to a radially expansive device for providing access into a patient's body, kits that contain the radially expansive device, as well as methods for providing minimally invasive access into a patient's body. The radially expansive device may be used in conjunction with a balloon, bladder, or other expansive device, which can be used to expand the radially expansive device and allow access to the surgical site.

This application claims priority to U.S. Provisional Application 60/698,128 under 35 U.S.C. 119(e) filed Jul. 11, 2005, the entire contents of which are incorporated herein by reference.

### FIELD OF THE INVENTION

The present invention relates to a minimally invasive surgical instrument for retracting the tissue before, during, and/or after surgery. In one particular variation, the invention relates to an expandable tubular element that retracts the skin by expanding the tubular element. The tubular element can be manipulated and thus, expanded by a mechanical device or by a person.

### BACKGROUND OF THE INVENTION

Patients who undergo minimally invasive surgery generally benefit from significantly less surgical trauma and post-operative pain when compared to open surgery. Patients who undergo minimally invasive surgery show other benefits compared to open procedures, such as considerable reduction in the use of narcotic pain medications, less blood loss and reduced hospital stay(s). All of these factors reduce hospital costs.

One of the minimally invasive surgical techniques that has led to decreased recovery time in patients, less reliance on narcotic drugs, and shorter hospital stays has been the use of a small access portal to the subcutaneous layers in a patient. This access portal is generally a small incision made in the skin in close proximity to the surgical site. Once the incision has been made, a variety of surgical instruments can pass through this access portal to perform procedures at the surgical site. Cannulae have been developed that allow unimpeded passage of surgical instruments from the outside of a patient to the inside. However, inserting a cannula often proves to be problematic due to the elasticity of the skin. After an incision is made, the skins elasticity seals the incision site, which precludes the facile passage of cannulae and/or surgical instruments.

Retractor clamps that have conventionally been used to retract the skin suffer from a series of drawbacks. These drawbacks include requiring many hands to operate the clamps to keep the skin retracted, bulkiness of the clamps, which may impede surgical access, and tearing of tissue and/or skin because of an inability to easily regulate the force by which skin/tissue is retracted. The tearing of skin and tissue makes surgery more invasive, which in turn minimizes the benefits of minimally invasive surgery alluded to above (i.e., reduction in the use of narcotic pain medications, less blood loss and reduced hospital stay(s)).

A surgeon's hand(s) has/have also been used to retract skin. However, when a surgeon uses his hand(s), the surgeons hand(s) is no longer free to perform other surgical procedures. If he moves his hand to grab a surgical instrument the skin is likely to close the incision due to the elasticity of the skin. Moreover, if the skin is not adequately retracted, it is difficult to move surgical instruments to the surgical site as the skin's elasticity (when it closes) creates a barrier. It is with these drawbacks in mind that the surgical instrument(s) of the instant invention was/were developed.

### BRIEF SUMMARY OF THE INVENTION

The instant invention relates to a minimally invasive surgical instrument for retracting the skin before, during, or after surgery. Moreover, the present invention provides skin retractor devices, systems, and methods for skin retraction in order to create a space at an incision site to improve access during minimally invasive surgery. In one particular variation, the invention relates to an expandable tubular element that retracts the skin by radially expanding the tubular element allowing access to a surgical site. The tubular element can be radially expanded by a mechanical device or by a person.

The tubular element is generally inserted in a non expanded state into a relatively small minimally invasive incision whereupon by mechanical means or some other means the tubular element is radially expanded in a controlled manner to retract the skin. In one variation, the tubular element is able to remain in an expanded state by the use of shape memory materials to keep the skin retracted and prevent the skin's elasticity from interfering with surgical access. Once the tubular element is in its radially expanded state, surgical tools have access to the underlying tissue, muscle, tissue fibers and/or surgical site below. In one particular design, the tubular element in its radially expanded state is configured to obtain a diameter of sufficient size so that the diameter of the tubular element can accommodate a plurality of types of surgical instruments.

Although the invention is described with reference to human patients, it should be understood that the invention can be used on animals and/or used for veterinarian purposes. Thus, when the word “patient” is used, it is contemplated that “patient” encompasses all animals (including humans) that may undergo surgical procedures.

### BRIEF DESCRIPTION OF THE SEVERAL VIEWS OF THE DRAWING

FIG. 1A discloses a small incision in the skin to access a surgical site and FIG. 1B shows one variation of the tubular radially expansive surgical instrument 1 with a balloon, bladder or expansive device 3 that is present in the tubular radially expansive surgical instrument 1. The balloon, bladder or expansive device 3 is filled with a liquid, solid, and/or gas that radially expands the tubular radially expansive surgical instrument 1.

FIG. 2 shows the tubular radially expansive surgical instrument 1 with the balloon, bladder or expansive device 3 removed. The tubular radially expansive surgical instrument 1 is in the expanded position and is retracting the skin.

FIG. 3 shows the insertion of a cannula 6 in the tubular radially expansive surgical instrument 1.

FIG. 4 shows a balloon, bladder or expansive device 3 being inserted into a cannula 6 wherein the balloon, bladder or expansive device 3 can be used for surgical procedures after proceeding through the cannula 6.

FIG. 5 shows a balloon, bladder or expansive device 3 that has passed through the cannula 6 and can be used for a surgical procedure.

### DETAILED DESCRIPTION OF THE INVENTION

The present invention relates to a minimally invasive surgical instrument for skin retraction, kits related to these surgical instruments, and methods and processes relating thereto. In one embodiment of the invention, the device comprises a tubular element that can be radially expanded. Upon radial expansion, the tubular element can stay in its expanded state, which when inserted into an incision through the skin is able to retract the skin (and at all times remaining in the expanded state). In another embodiment, the device is inserted into the incision in the compressed state, then expanded radially to expand the incision opening. A tubing or scaffolding may then be left in the incision opening to keep the incision opening in the expanded state while the rest of the device is retrieved to provide access to the surgical site through the expanded opening. In one application, the device is expanded to stretch the skin around the incision area to maximize the opening at the incision site. It will be appreciated that although the present invention is ideally suited for minimally invasive surgery, there are situations wherein the radially expansive surgical instrument for skin retraction could be used for open surgery.

Skin as used in the present invention includes one or more layers of the skin including the epidermal layer, the dermal layer and the hypodermal layer (including any adipose tissue that may lie below the hypodermal layer). Although the present invention was designed to provide means of retracting skin, the present invention may also be applicable in other surgical applications.

The tubular radially expansive surgical instrument 1 of the instant invention provides a quicker, more convenient way of retracting skin allowing better passage into a surgical site and may also provide better visualization of the surgical site. The tubular radially expansive surgical instrument 1 is ideally suited for minimally invasive surgical procedures to be performed wherein skin and/or tissue damage is minimized, post-operative recuperation is more rapid, and costs are lowered (due to shorter hospital stays and faster recovery times).

The present invention also relates to methods and a surgical system of extracting skin before, during and after surgery by using the tubular radially expansive surgical instrument 1 of the instant invention. In one variation, the method and surgical system comprises using a mechanical means and/or some other means of expanding the tubular radially expansive surgical instrument 1 to retract skin, and then removing the means of expanding the tubular radially expansive surgical instrument 1. In the method and surgical system of retracting the skin, the tubular radially expansive surgical instrument 1 stays in an expanded state conformation that allows the passage of surgical tools so they can arrive at or proceed towards the surgical site.

The present invention is described with reference to the figures, but the present invention should not be construed to limit the invention to the embodiments in the figures. It will be apparent to those of skill in the art that variations, modifications, changes, and adaptations of the present invention can be made without departing from the scope and spirit of the present invention. It should be apparent to those of skill that methods and surgical systems of using the tubular radially expansive surgical instrument 1 of the instant invention are contemplated and within the scope of the present invention.

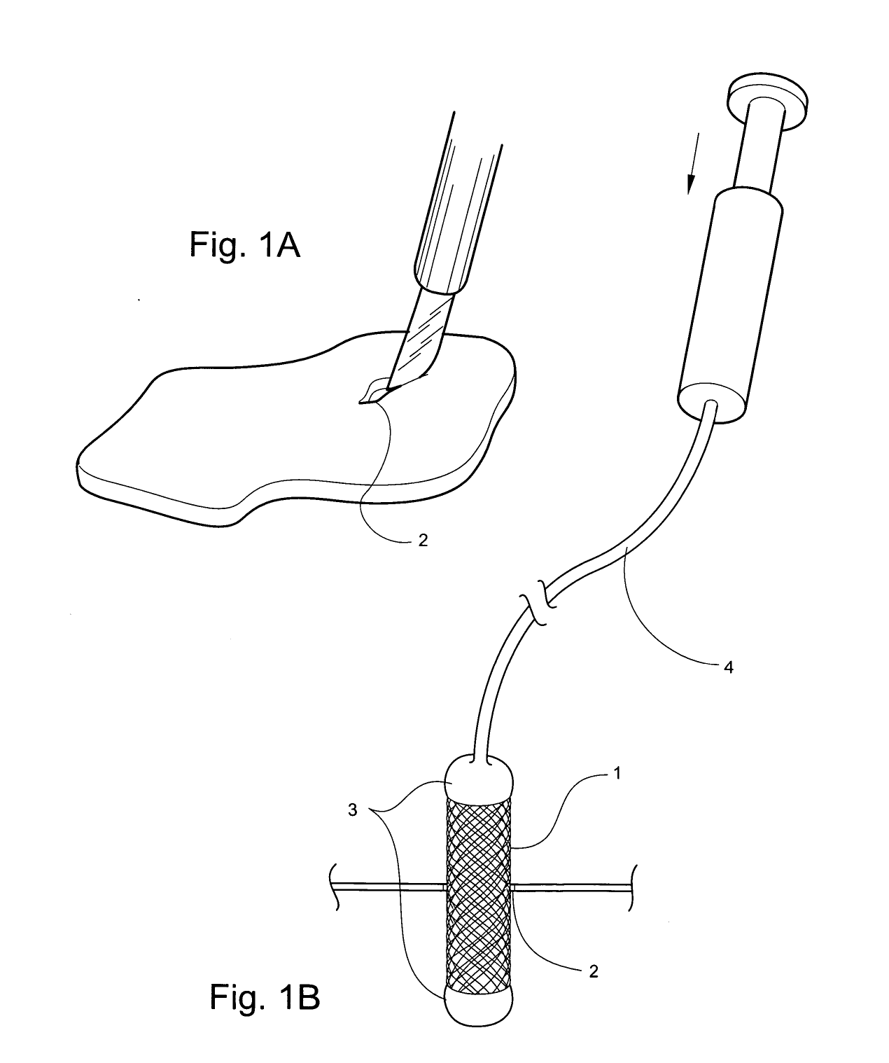

FIG. 1A shows an incision in the skin 2. In one embodiment, the incision is made to transverse the depths of the epidermal layer, the dermal layer and the hypodermal layer. In one application, the length of the incision is determined by the expanded outer diameter size of the tubular radially expansive surgical instrument 1 that is to be inserted through the skin at the incision site. The tubular radially expansive surgical instrument 1 has an inner diameter and an outer diameter. In one configuration, the tubular radially expansive surgical instrument 1 is generally tubular in shape, as a consequence, the cross sectional area of the tubular radially expansive surgical instrument 1 is roughly circular. Thus, the inner diameter of the tubular radially expansive surgical instrument 1 is the length from one side of the inside of the tubular radially expansive surgical instrument 1 to the inside of the other side of the tubular radially expansive surgical instrument 1 (which passes through the center of a cross-sectional area of the tubular radially expansive surgical instrument 1). The outer diameter is the distance from the outside of the tubular radially expansive surgical instrument 1 to the outside of the other side of the tubular radially expansive surgical instrument 1 (which passes through the center of a cross-sectional area of the tubular radially expansive surgical instrument 1). The inner diameter is configured with sufficient size so as to accommodate the surgical instruments that are going to be used at the surgical site. Accordingly, the expanded diameter size that the tubular radially expansive surgical instrument 1 is to attain is dependent on the types of surgical instruments that the surgeon plans to implement, and may also be dependent upon the type of surgery that is to be performed. One of ordinary skill in the art, having the benefit of this disclosure, would appreciate that the expansive surgical instrument 1 can be designed to form various shapes (e.g., oval, pentagonal, etc.) to provide access to the interior of the body.

For example, if minimally invasive back surgery is to be performed, preferably the surgical tool that has the widest cross sectional area width will determine the size that must be obtained by the expanded inner diameter of the tubular radially expansive surgical instrument 1. In one instance, a cannula may have the widest cross sectional area width. In this instance, the cannula will determine the width to be obtained by the tubular radially expansive surgical instrument 1 (because the tubular radially expansive surgical instrument 1 should be able to accommodate the insertion of the cannula through it). It should be noted that the size of the cannula (and in particular, the inner diameter of the cannula) is in turn determined by the size(s) of the surgical instruments that are to be used. The inner diameter of the cannula should be able to easily accommodate the surgical tools that are used, such as balloons, bladders, expandable elements, endoscopes, scalpels, screws (e.g., pedicle screws), plates, discs, and other tools that are used for back surgery. It is also possible that another surgical instrument may have the widest cross sectional area, such as an expansible tissue distractor. Thus, the expanded inner diameter of the tubular radially expansive surgical instrument 1, in this instance, should be able to accommodate passage of an expansible tissue distractor. An example of an expansible tissue distractor is disclosed in U.S. patent application Ser. No. 11/021,786 filed on Dec. 22, 2004, which is herein incorporated in its entirety by reference for all purposes. Moreover, all references referred to in U.S. application Ser. No. 11/021,786 are hereby also incorporated by reference in their entirety.

The length of the incision may be determined by the outer diameter of the expanded diameter of the tubular radially expansive surgical instrument 1, which in turn is determined by the inner diameter of the tubular radially expansive surgical instrument 1 (allowing access of instruments to the surgical site). Because the distance from the inner surface of the tubular radially expansive surgical instrument 1 to the outer surface of the tubular radially expansive surgical instrument 1 is known, the incision length can be calculated. In some applications, it is preferable that the incision length be roughly equivalent to the outer diameter of the tubular radially expansive surgical instrument 1 in the expanded state. Roughly matching the length of the incision to the outer diameter of the expanded state of the tubular radially expansive surgical instrument 1 may prevent complications. In certain applications, if the incision length is too short, tearing of the skin and/or underlying tissue can occur when the tubular radially expansive surgical instrument 1 is inserted at the incision site. If the incision length is too long, recuperative time, pain, and blood loss may increased relative to a shorter incision length (i.e., the surgery is less minimally invasive). Preferably, an incision length of from about 0.5 to about five centimeters or from about one centimeter to about five centimeters in length is made. Alternatively, the incision can be from two to four centimeters or from 2.5 to four centimeters or from 2.5 to three centimeters in length. It, however, should be understood that the incision can be of any length and is usually determined by the surgical instruments and the particular medical procedure to be implemented.

In one variation, the tubular radially expansive surgical instrument 1 when expanded obtains an outer diameter length that is roughly the same as the incision length. Thus, the outer diameter of the tubular radially expansive surgical instrument 1 when expanded is generally in the range of 0.5 to five centimeters, or one to five centimeters with other ranges being from two to four centimeters or from 2.5 to four centimeters or from 2.5 to three centimeters. However, it should be recognized by those of skill in the art that the size of the incision opening defined by the radially expansive surgical instrument 1 can be modified to meet the specific requirements of the medical procedure to be performed.

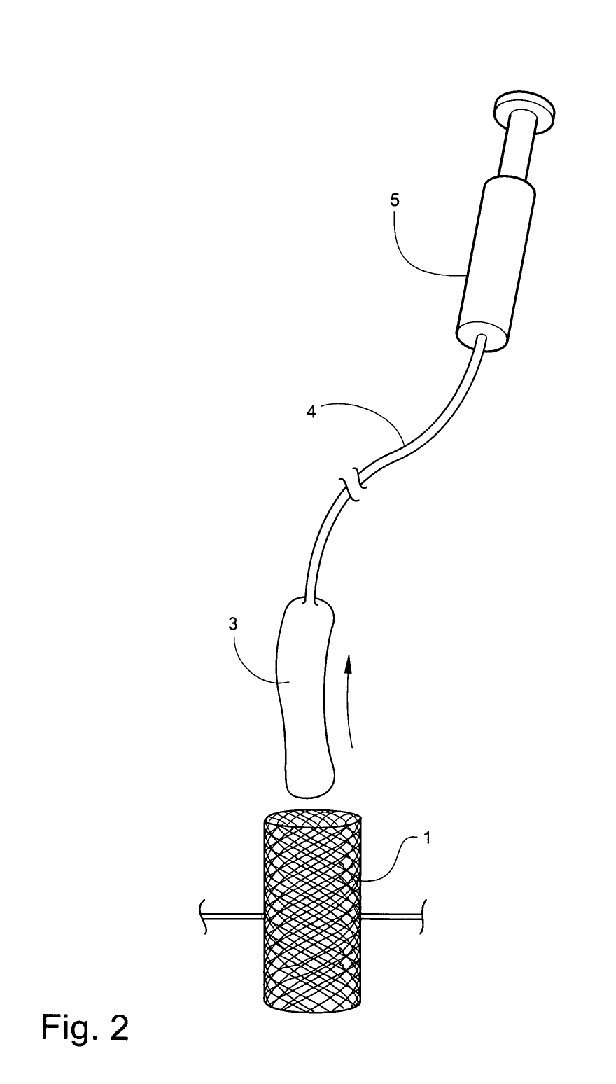

FIG. 1B shows the insertion of the tubular radially expansive surgical instrument 1 into the incision site 2 prior to expansion. In this embodiment, a balloon, bladder or expansive device 3 is inserted into the unexpanded tubular radially expansive surgical instrument 1 in preparation for expansion. The balloon, bladder or expansive device 3 can be inserted into the unexpanded tubular radially expansive surgical instrument before, during or after insertion of the tubular radially expansive surgical instrument into the skin. The balloon is connected to a tubular elongate member 4 that is in fluid connection with the balloon and a valve 5 so that a fluid, gas or solid can flow from the valve 5 through the elongate member 4 to the balloon, bladder or expansive device 3 to expand the balloon, bladder or expansive device 3. The expansion of the balloon, bladder or expansive device 3 expands the tubular radially expansive surgical instrument 1 to the correct size (as shown in FIG. 2).

The balloon, bladder or expansive device 3 can be formed from non-compliant, semi-compliant, compliant materials or any combination thereof. The balloon, bladder or expansive device 3 can be formed from a variety of medical grade materials including, but not limited to, TEXIN®, polyurethane, polyethylene, polyethylene terephthalate, polytetrafluoroethylene, nylon, silicone, latex, polyvinyl chloride, thermoplastic elastomer, elastic materials, and combinations thereof. In one variation, the balloon, bladder or expansive device 3 comprises a compliant material, such as TEXIN®. The balloon, bladder or expansive device 3 can be filled with any of a variety of materials including a gas (e.g., CO₂, N₂, Ar or other inert gas), fluid, (e.g., water, saline, dextrose water) radiopaque medium, silicone, gels, solid materials, and combinations thereof. Fluid is a medium that may be preferable because fluid flow can be precisely regulated so as to exert a measured force on the tubular radially expansive surgical instrument 1. By exerting this measured force the tubular radially expansive surgical instrument 1 can be expanded as quickly or as slowly as desired. Preferably, the tubular radially expansive surgical instrument 1 would be expanded at a rate wherein skin and tissue tearing is kept to a minimum. Further, the use of fluid reduces the possibility of explosions or rapid decompression of the balloon bladder or expansive device (which may happen when volatile gases are used). Moreover, a fluid can be carefully chosen so as to increase visualization and to reduce problems that result from rupture. For example, a balloon, bladder or expansive device 3 can be filled with a radiopaque solution to allow visualization by fluoroscopy or alternatively, the balloon, bladder or expansive device 3 can be filled with a biocompatible fluid, such as water or saline, such that in the event of accidental fluid leakage or rupture, contamination to the surgical area is minimized. Radiopaque solutions may be useful in certain applications to visualize the site at which surgery is to be performed, or for example, once the expansive device is expanded to make sure that the trajectory of the tubular radially expansive surgical instrument 1 is correct.

The tubular radially expansive surgical instrument 1 can be made from any one or more of a plurality of different materials. Preferably, a biocompatible material is used. These materials include metal alloys and/or polymers. Possible materials include, but are not limited to, nickel titanium alloys (e.g., NITINOL®), stainless steel (e.g. an interlocking stainless steel material with shape memory characteristics), expandable, wire-mesh tube, silicone and metal materials, superelastic and martensitic materials, tantalum, platinum, and titanium, niobium alloys, PHYNOX®, or any of a plurality of polymeric materials, such as expanded polytetrafluoroethylene (ePTFE). Generally, intraluminal stent-type materials can be used for making the tubular radially expansive surgical instrument 1, such as the stents that are described in U.S. Pat. No. 5,749,880 to Banas et al. (as well as stent patents that are referred to therein). U.S. Pat. No. 5,749,880 to Banas et al. and all patents referred to therein are hereby incorporated in their entirety by reference for all purposes. In an embodiment, materials are used that have shape memory characteristics that allow the tubular radially expansive surgical instrument 1 to retain its shape once it is in the expanded state.

In one variation, stent material is utilized in the fabrication of the tubular radially expansive surgical instrument 1 of the instant invention. Stent materials may be particularly applicable for certain applications since they have the structural integrity to keep intraluminal passageways open (for examples, in veins and/or arteries). It is also contemplated, other materials (that are not used in stents) which are sufficiently rigorous for the intended purpose of maintaining an opening at the incision site may also be applicable.

It is contemplated and also within the scope of the instant invention to use heat activated shape memory alloys and/or polymers for the tubular radially expansive surgical instrument 1. In an embodiment, warmer flowing material could be sent to the balloon, bladder or expansive device which would allow the use of, for example, a thermosetting resin for the tubular radially expansive surgical instrument 1, which would retain its shape after heating. Alternatively, an external heat source could be used to set the tubular radially expansive surgical instrument 1 at the desired diameter. For example, in one design variation, an electrical powered device (e.g., battery powered heater, direct current heating, etc.) is used to heat the incision and keep a resin on the expansive surgical instrument in a desired shape. When the heat is removed, the tubular radially expansive surgical instrument 1 may revert back to its unexpanded state.

It is also contemplated and within the scope of the invention to have heat expandable materials in the tubular radially expansive surgical instrument 1. The application of heat to the tubular radially expansive surgical instrument 1 will expand the tubular radially expansive surgical instrument 1 to the desired diameter. The diameter size of the expanded tubular radially expansive surgical instrument 1 can be determined by how the tubular radially expansive surgical instrument 1 is manufactured (e.g., the application of heat will expand the tubular radially expansive surgical instrument 1 to an enlarged diameter, wherein the diameter is controlled by the compositional materials and their amounts in the tubular radially expansive surgical instrument 1) or alternatively, by regulation at a constant given temperature (which will expand the tubular radially expansive surgical instrument 1 a given amount). In light of the above discussion, it should be apparent to one of ordinary skill in the art that there are a plurality of methods and systems for expanding the tubular radially expansive surgical instrument 1 to a given desired diameter. Alternatively, heat may revert the tubular radially expansive surgical instrument to its unexpanded shape.

The length of the tubular radially expansive surgical instrument 1 is sufficient so as to traverse the skin layers and the diameter of the tubular radially expansive surgical instrument 1 is sufficient so as to be able to accommodate the surgical instruments that are to be inserted into the tubular radially expansive surgical instrument 1. Preferably, the length of the tubular radially expansive surgical instrument 1 will be on the order of 1-15 cms. However, it should be understood that any length can be used (e.g., 1, 2,3, 4, 5, 6, 7, 8, 9, 10, 11, 12, 13, 14, or 15 cm or lengths that are longer). In some variations, the inner diameter of the tubular radially expansive surgical instrument 1 is configured to accommodate cannulae. Generally, cannulae will have outer diameters that range from about 1 cm to about 3.5 cm, or from about 2 cm to about 2.5 cm. In one design variation, the inner diameter of the tubular radially expansive surgical instrument 1 when expanded is on the order of the outer diameter of a cannula.

The elongate member 4 may be made from a resilient inert material providing torsion transmission capabilities (e.g., stainless steel, a nickel-titanium alloy such as NITINOL®, and other suitable metal alloys). In other embodiments, the elongate member 4 may be fashioned from a variety of suitable materials, comprising a carbon fiber, a glass, or a flexible material, such as a plastic or rubber. In one embodiment comprising a flexible elongate member 4, the elongate member 4 may be, for example, fashioned from twisted wire filaments, such as stainless steel, nickel-titanium alloys (such as NITINOL®), and suitable other metal alloys.

The elongate member 4 shown is hollow, allowing movement of a flowable material within a bore therethrough along its axis. A flowable material may comprise, for example, a liquid material, a gaseous material, a slurry, a sludge, a plasma, a paste, a flowable solid (such as powdered, pulverized, granulated, pelletized, or encapsulated material), or any other suitable material that may flow naturally or be made to flow from one place to another. Alternatively, the elongate member 4 can be manufactured from a flexible polymeric material, such as the materials that are commonly used in catheters.

In some variations, the elongate member 4 is attached to a valve 5 which allows for the flow of a flowing material such as a liquid material, a gaseous material, a slurry, a sludge, a plasma, a paste, or a flowable solid. In one embodiment, the valve 5 may have dual valves with one valve allowing the passage of the flowing material into the balloon, bladder or expansive device 3. A second valve may be present which allows the flow of the flowing material from the balloon, bladder or expansive device 3. Ideally, the valve(s) will allow a measured flow of the flowing material to and from the balloon, bladder or expansive device 3, so that the pressure in the balloon, bladder or expansive device 3 can be precisely controlled. Precise control allows the tubular radially expansive surgical instrument 1 to be expanded in a measured manner so that skin and/or tissue tearing is kept to a minimum. Moreover, careful control of the flowing material will allow the tubular radially expansive surgical instrument 1 to obtain the precisely desired diameters.

One or more gauges may be optionally operatively connected to one or more of the valves, to the elongate member 4, or to any other position on the balloon, bladder, or expansive device so as to precisely measure and/or quantitate flow rates and volumes of the flowing material as it goes in to or out from the balloon, bladder, or expansive device 3. As the flowing material enters the balloon, bladder, or expansive device 3, it expands the tubular radially expansive surgical instrument 1 to the desired size. In some variations, volumes of flowing material that may be used to fill the balloon, bladder, or expansive device 3 are in a range from about 3 cc to about 30 cc, or in range from about 10 cc to about 18 cc. It should be understood that the volume that is used is determined by the cross sectional area that is desired by the tubular radially expansive surgical instrument 1 to allow passage of surgical instruments.

In FIG. 1B the tubular radially expansive surgical instrument 1 is shown being expanded by a balloon, bladder or expansive device 3. The balloon, bladder, or expansive device 3 should be properly positioned in the tubular radially expansive surgical instrument 1 so as to allow optimal expansion. In some variations, the inner diameter of the tubular radially expansive surgical instrument 1 in its unexpanded state is sufficiently large so as to accommodate the balloon, bladder, or expansive device 3, so the balloon, bladder, or expansive device 3 can be positioned within the tubular radially expansive surgical instrument 1 relatively easily. When the balloon, bladder, or expansive device 3 is being expanded, the tubular radially expansive surgical instrument 1 is also being expanded which pushes the skin outwards in a radial direction. Preferably, the flow rate of the flowing material into the balloon, bladder or expansive device 3 is carefully controlled so as to minimize tearing of skin and tissue.

In some variations, other means of expanding the tubular radially expansive surgical instrument 1 can be employed such as by mechanical means, such as a person's (e.g., physician, nurse, physicians assistant, etc.) hand or fingers, a stylet, clamps, or any of a plurality of other means. Moreover, in FIG. 1B, the particular variation of the balloon, bladder or expansive device 3 shown is longer than the tubular radially expansive surgical instrument 1. In an embodiment, the balloon, bladder or expansive device 3 transverses the full length of the tubular radially expansive surgical instrument 1 so that the balloon, bladder or expansive device 3 is uniformly inflated. If the balloon, bladder or expansive device 3 does not transverse the full length of the tubular radially expansive surgical instrument 1, the tubular radially expansive surgical instrument 1 may end up with a roughly conical shape (by having the balloon, bladder or expansive device 3 positioned at the proximal or distal end of the tubular radially expansive surgical instrument 1) or alternatively, a barrel shape if the balloon, bladder or expansive device 3 is shorter in length than the tubular radially expansive surgical instrument 1 and the balloon, bladder, or expansive device 3 is inflated in the central portion of the tubular radially expansive surgical instrument 1. All of these various shapes are contemplated as being part of the invention. In an embodiment wherein the balloon, bladder or expansive device is uniformly inflated, the user centers the balloon, bladder, or expansive device 3 around the center of the length of the tubular radially expansive surgical instrument 1 to generate a uniformly expanded tubular radially expansive surgical instrument. Centering the balloon, bladder, or expansive device 3 may be useful in certain applications since some variations of the balloons, bladders and expansive devices also have tips that curve up or down, which can potentially lead to inflation problems.

After expansion of the balloon, bladder, or expansive device 3 to an appropriate level that expands the tubular radially expansive surgical instrument 1 to the desired size, the flow material can then be withdrawn from the balloon, bladder, or expansive device 3. The tubular radially expansive surgical instrument 1 material composition will allow the tubular radially expansive surgical instrument 1 to remain at the expanded size when the balloon, bladder, or expansive device 3 is deflated. When the balloon, bladder, or expansive device 3 has reached sufficient deflation, the balloon, bladder or expansive device 3 can be removed.

FIG. 2 shows the balloon, bladder or expansive device 3 being removed from the tubular radially expansive surgical instrument 1 once it has been expanded to the desired diameter to retract the skin and allow surgical instruments passage to the surgical site or alternatively to the underlying tissue. The tubular radially expansive surgical instrument 1 can then accommodate one or more surgical instruments, including the expansible tissue distractor, which is disclosed in U.S. application Ser. No. 11/021,786 and is hereby incorporated by reference in its entirety. The variety of surgical instruments that can pass through the tubular radially expansive surgical instrument 1 may be limited by the size of the diameter opening. Surgical instruments that can pass through the tubular radially expansive surgical instrument 1 include balloons, bladders, expandable elements, forceps, retractors, scissors, probes, stylets, knives, speculas, needles, clamps, speculae, hammers, suction tubes, spreaders, razors, hooks, pins, resection tools, clips, catheters, endoscopes, scalpels, screws (e.g., pedicle screws), plates, discs, cannulae, and other tools.

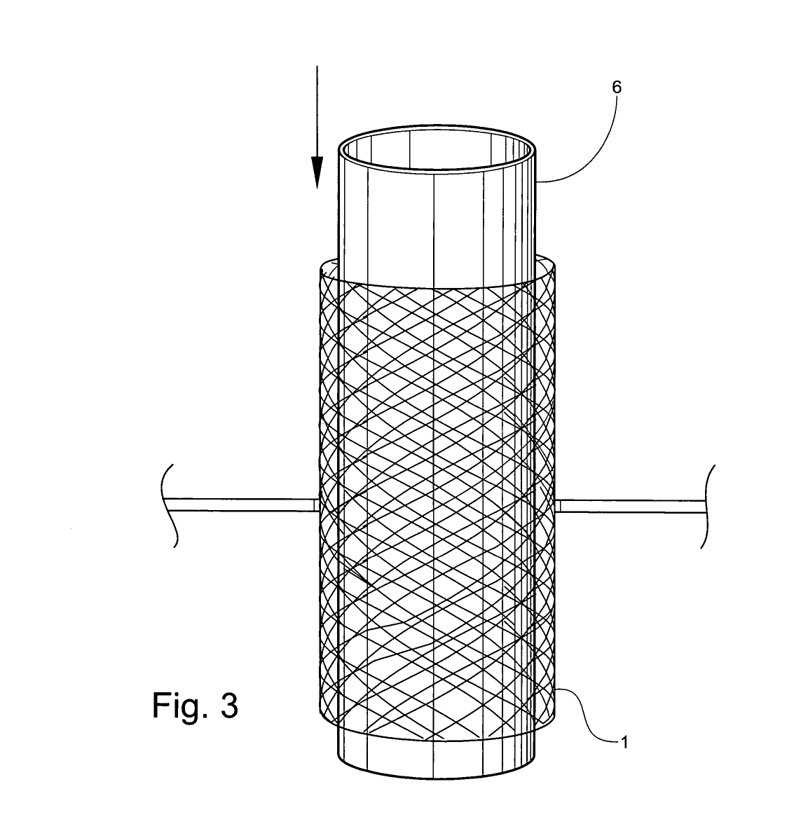

FIG. 3 shows a cannula 6 being inserted through the tubular radially expansive surgical instrument 1. The cannula 6 has an outer diameter which fits into the inner diameter of the tubular radially expansive surgical instrument 1. The insertion of the cannula 6 into the tubular radially expansive surgical instrument 1 allows passage of surgical instruments to depths below that which may not be obtainable with the tubular radially expansive surgical instrument 1 alone. This cannula 6 is able to accommodate the passage of a plurality of surgical instruments, such as balloons, bladders, expandable elements, forceps, retractors, scissors, probes, stylets, knives, speculas, needles, clamps, speculae, hammers, suction tubes, spreaders, razors, hooks, pins, resection tools, clips, catheters, endoscopes, scalpels, screws (e.g., pedicle screws), plates, discs, and other tools.

One can employ radially expansive cannulae 6 that expand only in one cross sectional area of the length of the cannulae 6, or alternatively, one can employ cannulae 6 that can expand radially along their entire lengths. Alternatively, one can employ cannulae 6 that expand along portions of their lengths. Expansion of the cannulae 6 can occur against the viscoelastic resistance of the surrounding tissue. The expandable cannulae 6 do not require a full depth incision, and generally at most require only a needle-size entrance opening. However, the presence of the tubular radially expansive surgical instrument 1 already transverses and retracts the skin allowing easy passage of an expandable or non-expandable cannulae 6. The cannula 6 may be expanded by inserting members or by fluid pressure. An expandable chamber may be provided at the distal end of the cannula 6.

When a cannula 6 is being used in conjunction with the expansible tissue distractor as described in U.S. application Ser. No. 11/021,786 (which is incorporated by reference in its entirety), one may use the expansible tissue distractor to distract soft tissue and then shuttle the cannula 6 to deeper surgical depths (through the tissue). This can be an iterative process where the distractor is used and the cannula 6 is moved, then the distractor is used and the cannula 6 is moved, etc. If there is no tubular radially expansive surgical instrument 1 present in the expanded state, moving the cannula 6 often proves to be quite difficult as the skin tends to grip the cannula 6 thereby impeding its entrance to the surgical site. (proximal end outside skin)

Although FIG. 3 shows the insertion of a cannula 6 through the tubular radially expansive surgical instrument 1, it should be understood that any of a plurality of instruments can be inserted through the tubular radially expansive surgical instrument 1. The presence of the tubular radially expansive surgical instrument 1 effectively retracts the skin and allows passage to the lower layers of the skin and/or the underlying soft tissue.

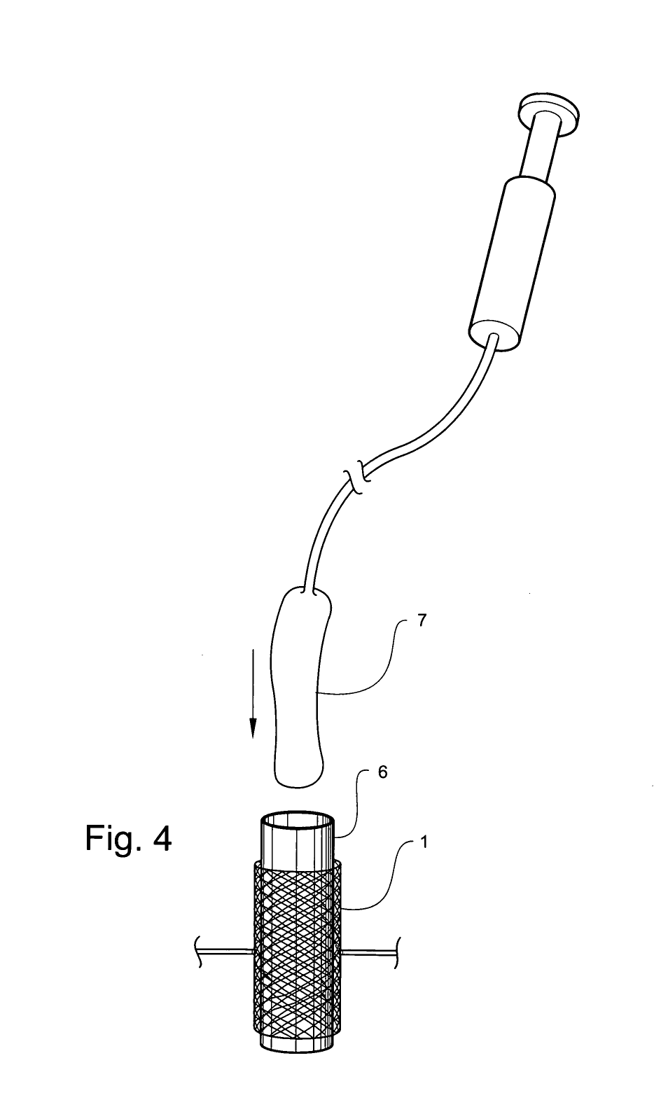

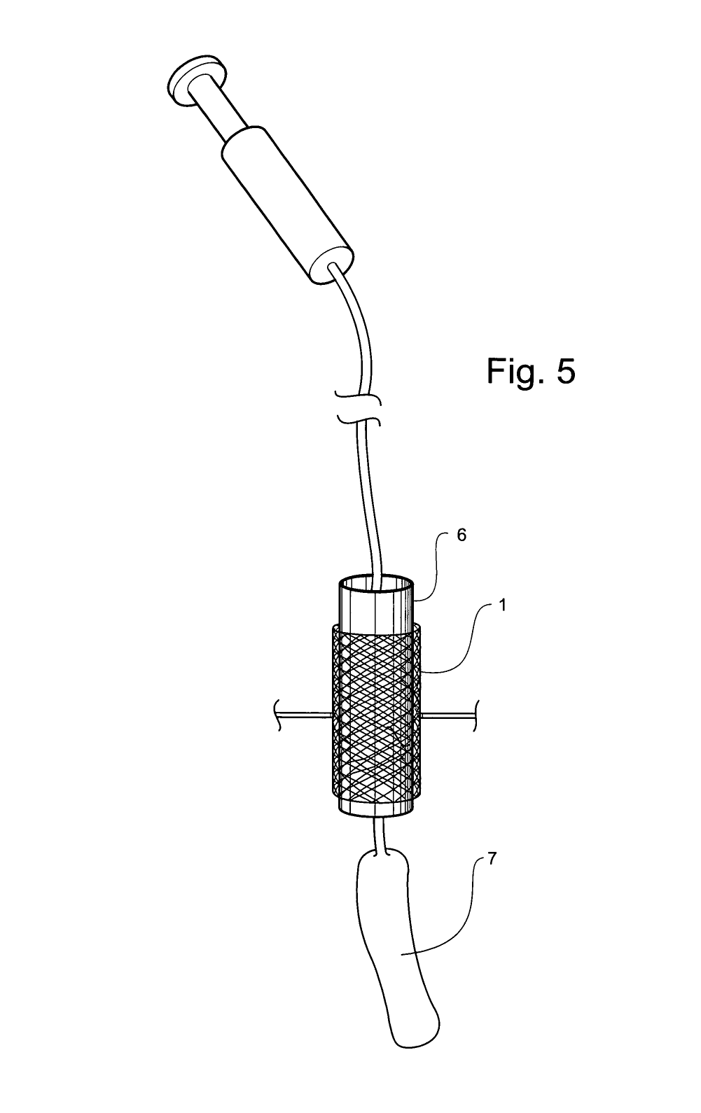

FIGS. 4 and 5 show the insertion of a balloon, bladder or expansive device 7 through the cannula 6. In one embodiment, the balloon, bladder or expansive device 7 passing through the cannula 6 can be used for surgery on the spine such as disclosed in U.S. Pat. No. 6,899,719 to Reiley, et al. which is hereby incorporated by reference in its entirety for all purposes.

After surgery is completed or at any time before or during the surgical procedure, the tubular radially expansive surgical instrument 1 can be removed. Because the skin is elastic and the incision used in the minimally invasive surgery is small, the site of the incision would likely require many fewer stitches than are required when conventional open surgery is done.

In one variation, the minimally invasive surgical instrument comprises a radially expansive tubular element, wherein the radially expansive tubular element is inserted in an incision in a patient's skin and upon expansion of the tubular element, the tubular element retracts the patient's skin.

In an embodiment, the minimally invasive surgical instrument further comprises a member selected from the group of a balloon, bladder, and an expansive device.

In another embodiment, the minimally invasive surgical instrument has a radially expansive tubular element that is expanded to an outer diameter that is about one to five centimeters in length.

In another embodiment, the minimally invasive surgical instrument has a radially expansive tubular element that is expanded to an outer diameter that is about 2.5 to five centimeters in length.

In an embodiment the minimally invasive surgical instrument has a radially expansive tubular element that is expanded to an outer diameter that is about 2.5 to five centimeters in length. In another embodiment, a method for providing access to a surgical site in a patient comprises inserting a radially expansive tubular element into an incision in the skin of said patient, and expanding said radially expansive tubular element.

In an embodiment, the method of the present invention further comprises retracting the skin.

In an embodiment, the method uses a radially expansive tubular element that has an outer diameter of from about one to five centimeters. The method may use a radially expansive tubular element that when expanded is any of 1.5, two, 2.5, three, 3.5, four, or 4.5 centimeters in its outer diameter.

In another variation, the method for retracting skin in a patient comprises inserting a radially expansive tubular element into an incision in the skin of said patient; and expanding the radially expansive tubular element.

In yet another variation, the invention comprises a surgical kit, wherein the kit comprises the radially expansive tubular element as described above.

In one variation, the invention is directed to a minimally invasive skin retracting surgical instrument that comprises a radially expansive tubular element, wherein the radially expansive tubular element is inserted in an incision in a patient's skin in an unexpanded state, and upon expansion of the tubular element, the tubular element retracts the patient's skin.

In an embodiment, the minimally invasive surgical instrument further comprises a member selected from the group of a balloon, bladder, and an expansive device.

In another embodiment, the minimally invasive surgical instrument has a radially expansive tubular element that can be expanded to an outer diameter that is about one to five centimeters in length or about 2.5 to five centimeters in length or about 2.5 to four centimeters in length or about 1.5 to 4.5 centimeters in length.

In an embodiment, the minimally invasive surgical instrument has a balloon, bladder, or expansive device that is of a size that allows the balloon, bladder, or expansive device to be inserted into the radially expansive tubular element. The balloon, bladder, or expansive device can expand the radially expansive tubular element.

In an embodiment, the minimally invasive surgical instrument is made of one or more members selected from the group consisting of nickel titanium alloys, NITINOL®, stainless steel, expandable, wire-mesh tube, silicone and metal materials, superelastic and martensitic materials, tantalum, platinum, and titanium, niobium alloys, and PHYNOX®.

In another embodiment, the balloon, bladder or expansive device of the minimally invasive surgical instrument is filled with one or more members selected from the group consisting of a gas, fluid, radiopaque medium, silicone, gels, solid materials, and combinations thereof.

In another embodiment, the instant invention is directed to a method of providing access to a surgical site in a patient comprising: inserting a radially expansive tubular element into an incision in the skin of the patient; and expanding the radially expansive tubular element to provide access to the surgical site.

In an embodiment, the method further comprises the step of using a balloon, bladder or expansive device to expand the radially expansive tubular element.

In another embodiment, the method involves expanding the radially expansive tubular element to a size that is between about 1.5 to 4.5 centimeters in diameter or about 2.0 to 4.0 centimeters in diameter. In an embodiment, employing the method retracts the skin.

One variation of the present invention is directed to a surgical kit comprising a radially expansive tubular element and a balloon, bladder or expansive device. In an embodiment the surgical kit further comprises a means of expanding the balloon, bladder, or expansive device. In an embodiment the surgical kit's means of expanding the balloon, bladder, or expansive device is selected from the group consisting of one or more members selected from the group consisting of a gas, fluid, radiopaque medium, silicone, gels, solid materials, and combinations thereof.

In an embodiment, the surgical kit further comprises a cannula.

It is contemplated and therefore within the scope of the invention that any time that a range is given, all values within that range are contemplated as potential end points for sub-ranges. For example if a range of 1 to 5 is recited, 2.5 to 4.3 are contemplated values for a sub-range (even though 2.5 to 4.3 may have not been explicitly recited). Moreover, although some features of the present invention are described with the surgical product and some features are discussed with relation to surgical methods or surgical systems or surgical kits, it should be understood that any of the features that are disclosed in the foregoing written description can be combined with any other feature as disclosed above.

Additionally, although certain exemplary embodiments and methods have been described in some detail, for clarity of understanding and by way of example, it will be apparent from the foregoing disclosure to those skilled in the art that variations, modifications, changes, and adaptations of such embodiments and methods may be made without departing from the true spirit and scope of the invention. Where methods and steps described above indicate certain events occurring in certain order, those of ordinary skill in the art will recognize that the ordering of certain steps may be modified and that such modification are in accordance with the variations of the invention. Furthermore, certain steps may be performed concurrently in a parallel process when possible, as well as performed sequentially as describe above.

Therefore, the above description should not be taken as limiting the scope of the invention but rather the invention should be defined by the below claims. Furthermore, a claim limitation should be interpreted to invoke 35 U.S.C. § 112, sixth paragraph, if and only if the claim limitation uses the phrases “means for” or “step for.” A claim limitation that does not include either of these phrases should be considered not to invoke 35 U.S.C. § 112, sixth paragraph.

We claim:

1. A tissue retracting surgical instrument that comprises:
a radially expansive tubular element, wherein the radially expansive tubular element is inserted in an incision in a patient's skin in an unexpanded state, and upon radial expansion of the tubular element, the tubular element retracts the patient's tissue.

2. The surgical instrument of claim 1, further comprising one or more members selected from the group consisting of a balloon, bladder, and an expansive device wherein said one or more members is used to expand the radially expansive tubular element.

3. The surgical instrument of claim 1, wherein the radially expansive tubular element can be expanded to an outer diameter that is about one to five centimeters.

4. The surgical instrument of claim 1, wherein the radially expansive tubular element is expanded to an outer diameter that is about 2.5 to five centimeters.

5. The surgical instrument of claim 4, wherein the radially expansive tubular element is expanded to an outer diameter that is about 2.5 to four centimeters.

6. The surgical instrument of claim 2, wherein the balloon, bladder, or expansive device is of a size that allows the balloon, bladder, or expansive device to be inserted into the radially expansive tubular element.

7. The surgical instrument of claim 6, wherein the balloon, bladder, or expansive device expands the radially expansive tubular element.

8. The surgical instrument of claim 7, wherein the minimally invasive surgical instrument expands to a size between about 1.5 to 4.5 centimeters in diameter.

9. The surgical instrument of claim 1, wherein the radially expansive surgical instrument is made of one or more members comprising nickel titanium alloys, NITINOL®, stainless steel, expandable, wire-mesh tube, silicone and metal materials, superelastic and martensitic materials, tantalum, platinum, titanium, niobium alloys, or PHYNOX®.

10. The invasive surgical instrument of claim 2, wherein the balloon, bladder or expansive device is filled with one or more members comprising a gas, fluid, radiopaque medium, silicone, gels, solid materials, and/or combinations thereof.

11. The surgical instrument of claim 1, wherein the tissue is skin.

12. The surgical instrument of claim 1, wherein the surgical instrument maintains shape after expansion.

13. A surgical instrument comprising a radially expansive tubular element made of a material that allows expansion of the radially expansive tubular element from an unexpanded state by a balloon, bladder, or expansive device to an expanded state; wherein when the radially expansive tubular element is in the expanded state and the balloon, bladder or expansive device is removed, the radially expansive tubular element maintains its shape allowing surgical instruments to pass from outside a patient to inside the patient through the expanded radially expansive tubular element.

14. The surgical instrument of claim 13, wherein the radially expansive tubular element in the expanded state has an outer diameter of between about 0.5 and about 5 cm.

15. The surgical instrument of claim 14, wherein after removal of the balloon, bladder, or expansive device from the radially expansive tubular element in the expanded state, the radially expansive tubular element reverts to the unexpanded state by application of heat.

16. The surgical instrument of claim 13, wherein the radially expansive tubular element is made of one or more members comprising nickel titanium alloys, NITINOL®, stainless steel, expandable, wire-mesh tube, silicone and metal materials, superelastic and martensitic materials, tantalum, platinum, titanium, niobium alloys, or PHYNOX®.

17. The surgical instrument of claim 14, wherein the radially expansive tubular element is made of one or more members comprising nickel titanium alloys, NITINOL®, stainless steel, expandable, wire-mesh tube, silicone and metal materials, superelastic and martensitic materials, tantalum, platinum, titanium, niobium alloys, or PHYNOX®.

18. The surgical instrument of claim 13, wherein the outer diameter of the radially expansive tubular element in the expanded state is uniformly expanded throughout a length of the expanded radially expansive tubular element.

19. The surgical instrument of claim 14, wherein the outer diameter is between about 2.5 and 5.0 cm.

20. The surgical instrument of claim 13, wherein a proximal end of the radially expansive tubular element is outside the patient.

21. A method of providing access to a surgical site in a patient comprising: inserting a radially expansive tubular element into an incision in the skin of said patient; expanding said radially expansive tubular element to provide access to the surgical site wherein expanding the radially expansive tubular element is by way of a balloon, bladder, or expansive device.

22. The method according to claim 21, wherein the radially expansive tubular element is expanded to a size that is between about 0.5 to 4.5 centimeters in diameter.

23. The method according to claim 21, wherein the radially expansive tubular element is expanded to a size that is between about 1.5 to 4.5 centimeters in diameter.

24. The method according to claim 22, wherein the radially expansive tubular element is expanded to a size that is between about 1.5 to 3.0 centimeters in diameter.

25. The method according to claim 21, further comprising retracting the skin.

26. The method according to claim 21, wherein the radially expansive tubular element is expanded to a size that is between about 2.0 to 4.0 centimeters in diameter.

27. A surgical kit comprising:
a radially expansive tubular element; and
a balloon, bladder, or expansive device, wherein the balloon, bladder, or expansive device is configured to be inserted into the radially expansive tubular element to expand the radially expansive tubular element, and the radially expansive tubular element when in an expanded state has an outer diameter of between about 0.5 to about 5 cm.

28. The surgical kit according to claim 27, further comprising a means of expanding the balloon, bladder, or expansive device.

29. The surgical kit according to claim 28, wherein the means of expanding the balloon, bladder, or expansive device is one or more members selected from the group consisting of a gas, fluid, radiopaque medium, silicone, gels, solid materials, and combinations thereof.

30. The surgical kit according to claim 27, further comprising a cannula.

31. The surgical kit according to claim 27, wherein the radially expansive tubular element when in an expanded state has an outer diameter of between about 1.0 to about 5 cm.
